(19) United States
(12) Patent Application Publication
(10) Pub. No.: US 2010/0217330 A1
(43) Pub. Date: Aug. 26, 2010

(54) BONE FIXATION DEVICES AND METHODS

(76) Inventors: Christopher U. Phan, San Leandro, CA (US); Darrel S. Brodke, Salt Lake City, UT (US)

Correspondence Address: MEDTRONIC;Attn: Noreen Johnson - IP Legal Department, 2600 Sofamor Danek Drive, MEMPHIS, TN, 38132, US

(21) Appl. No.: 12/391,672

(22) Filed: Feb. 24, 2009

(51) Int. Cl. A61B 17/58

(52) U.S. Cl. 606/301; 606/305; 606/308

(57) ABSTRACT

An apparatus includes a bone screw and a retention member. The bone screw has a proximal end portion and a distal end portion. The distal end portion of the bone screw includes a threaded portion. The retention member is coupled to the proximal end portion of the bone screw such that movement of the retention member relative to the bone screw is limited. The retention member is configured to deform when moved from a first configuration to a second configuration.

### BACKGROUND

The invention relates generally to medical devices and procedures. More particularly, the invention relates to apparatus and methods for inserting screws into bone tissue.

Bone fixation devices, such as, for example, bone screws, staples, and/or clamping mechanisms, can be used in various medical procedures. For example, known bone screws can be used to repair fractured bone tissue by clamping adjacent portions of the bone tissue together. Known bone screws can also be used to stabilize and/or limit the movement of bone tissue. For example, some known bone screws can be used as a part of a spinal fixation procedure.

In some procedures, for example, a facet screw can be inserted across the facet joint of the spinal column to fixate, fuse and/or limit the motion of the facet joint. Such known procedures can include, for example, translaminar facet screw fixation, which includes inserting a facet screw from the base of the spinous process on the contralateral side and through the lamina to traverse the facet joint along an axis perpendicular to the joint surfaces. Facet screws can also be inserted using a transfacet approach, which involves inserting a bone screw via a midline incision or an ipsilateral incision.

Such known procedures can further include coupling a retention member to the proximal end of the facet screw to fixate, fuse and/or immobilize the facet joint. Such retention members, however, can have a diameter larger than the diameter of the screw. This arrangement requires that the bone screw and the retention member be inserted via a cannula having a diameter at least as large as the diameter of the retention member.

Additionally, such known procedures often involve the use of multiple tools and/or multiple steps. For example, such known procedures can include separate steps and tools to advance a guide wire into the targeted bone tissue, insert the facet screw into the targeted bone tissue, and/or couple the retention member to the proximal end of the facet screw.

Thus, a need exists for improved insertion tools, bone fixation devices, and procedures for inserting bone screws into bone tissue.

### SUMMARY

In some embodiments, an apparatus includes a bone screw and a retention member. The bone screw has a proximal end portion and a distal end portion. The distal end portion of the bone screw includes a threaded portion. The retention member is coupled to the proximal end portion of the bone screw such that movement of the retention member relative to the bone screw is limited. The retention member is configured to deform when moved from a first configuration to a second configuration.

### BRIEF DESCRIPTION OF THE DRAWINGS

FIGS. 1 and 2 are schematic illustrations of an implant having a retention member in a first configuration and a second configuration, respectively, according to an embodiment.

FIGS. 3 and 4 are schematic illustrations of an implant having a retention member in a first configuration and a second configuration, respectively, according to an embodiment.

FIG. 5 is a schematic illustration of a tool used to deliver an implant into a body of a patient, according to an embodiment.

FIG. 6 is a front perspective view of a portion of an implant, according to an embodiment.

FIGS. 7-8 are front perspective views of an implant having a retention member in a first configuration and a second configuration, respectively, according to an embodiment.

FIG. 9 is a perspective view of a portion of a tool used to deliver the implant shown in FIGS. 7 and 8 into a body of a patient, according to an embodiment.

FIGS. 10, 11, and 12 are front perspective views of the implant of FIG. 7 connected to the tool of FIG. 9.

FIG. 13 is a cross-sectional view of the implant of FIG. 7 connected to the tool of FIG. 9, taken along the line X-X in FIG. 10.

FIG. 14 is a cross-sectional view of the implant of FIG. 7 connected to the tool of FIG. 9, taken along the line Y-Y in FIG. 12.

FIG. 15 is a flow chart illustrating a method of implanting an implant within a body of a patient, according to an embodiment.

### DETAILED DESCRIPTION

Apparatus and methods for inserting bone screws are described herein. In some embodiments, an apparatus includes a bone screw and a retention member. The bone screw has a proximal end portion and a distal end portion. The distal end portion of the bone screw includes a threaded portion. The retention member is coupled to the proximal end portion of the bone screw such that movement of the retention member relative to the bone screw is limited. The retention member is configured to deform when moved from a first configuration to a second configuration.

In some embodiments, an apparatus includes a first shaft, a second shaft and an actuator movably disposed about the second shaft. The first shaft has an engagement portion configured to engage a first engagement portion of a bone fixation device such that axial movement of the bone fixation device relative to the first shaft is limited in a distal direction. The second shaft has an engagement portion configured to engage a second engagement portion of the bone fixation device such that rotation of the second shaft results in rotation of the bone fixation device. The actuator is configured to deform a portion of the bone fixation device when the engagement portion of the first shaft is engaged with the first engagement portion of the bone fixation device and the engagement portion of the second shaft is engaged with the second engagement portion of the bone fixation device.

In some embodiments, a method includes inserting a bone fixation device into a body. The bone fixation device includes a bone screw and a retention member fixedly coupled to a proximal end portion of the bone screw. At least a distal end portion of the bone screw is disposed within a passageway defined by a bone tissue after the bone fixation device is inserted. The retention member is then deformed without moving the bone screw relative to the bone tissue. The step of disposing of the bone screw within the passageway is independent from the step of deforming the retention member.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to the direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a medical device first inserted inside the patient's body would be the distal end, while the opposite end of the medical device (e.g., the end of the medical device being operated by the operator) would be the proximal end of the medical device.

The term “parallel” is used herein to describe a relationship between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

The terms “perpendicular”, “orthogonal”, and/or “normal” are used herein to describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal to a curved surface when the line and the curved surface intersect at an angle of approximately 90 degrees within a plane. Two geometric constructions are described herein as being, for example, “perpendicular” or “substantially perpendicular” to each other when they are nominally perpendicular to each other, such as for example, when they are perpendicular to each other within a tolerance. Such tolerances can include, for example, manufacturing tolerances, measurement tolerances or the like.

It should be understood that the references to geometric constructions are for purposes of discussion and illustration. The actual structures may differ from geometric ideal due to tolerances and/or other minor deviations from the geometric ideal.

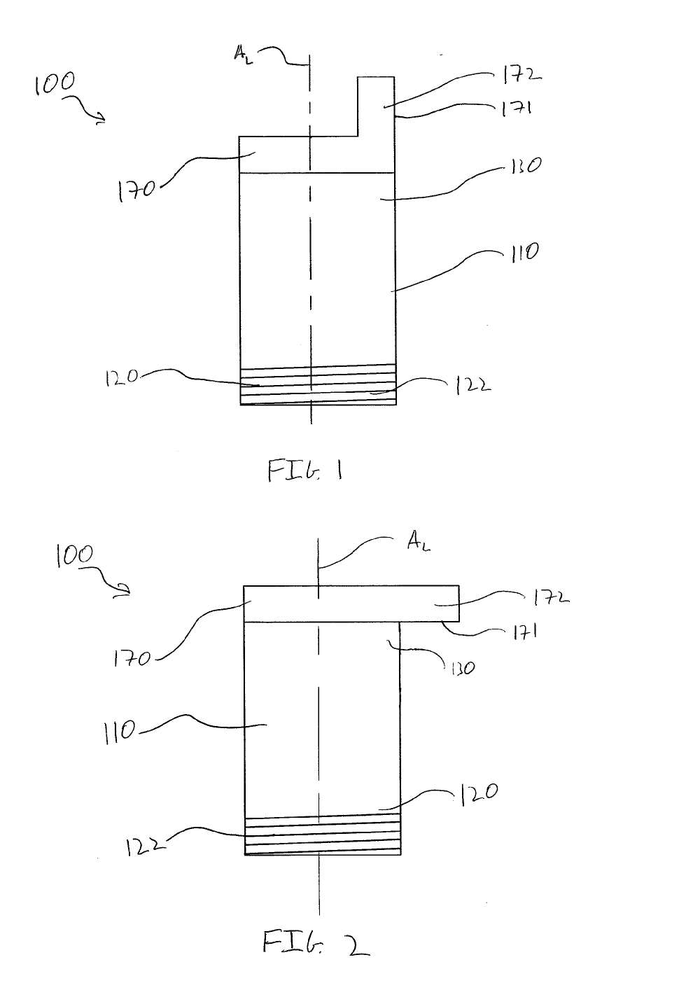

FIGS. 1 and 2 are schematic illustrations of an implant 100 having a retention member 170 in a first configuration and a second configuration, respectively, according to an embodiment. The implant 100 includes a bone screw 110 and a retention member 170. The bone screw 110 has a proximal end portion 130 and a distal end portion 120. In some embodiments, the bone screw 110 defines a lumen (i.e., the bone screw 110 can be cannulated for insertion over a guide wire, a K-wire or the like). The proximal end portion 130 of the bone screw 110 is coupled to the retention member 170 such that movement of the retention member 170 with respect to the bone screw 110 is limited and/or prevented. For example, in some embodiments, the retention member 170 can be fixedly coupled to the bone screw 110.

The distal end portion 120 of the bone screw 110 has a threaded portion 122. The distal end portion 120 is configured to be inserted into a bone tissue of a patient. As the bone screw 110 is rotated, the threaded portion 122 can be screwed into the bone tissue of the patient, as further described herein. In some embodiments, the threaded portion 122 of the bone screw 110 includes a self-tapping tip. Said another way, when rotated in a first direction (e.g., clockwise), the bone screw 110 can advance into a bone tissue without the aid of a previously defined hole or passageway in the bone tissue. In other embodiments, the threaded portion 122 of the bone screw 110 does not include a self-tapping tip.

The retention member 170 includes a deformable portion 172. The deformable portion 172 of the retention member 170 is configured to plastically deform when moved from a first configuration to a second configuration. Said another way, the deformable portion 172 of the retention member 170 is deformed such that the implant 100 remains in the second configuration.

When the retention member 170 is in the first configuration (FIG. 1), the retention member 170 has an outer diameter that is approximately equal to the outer diameter of the bone screw 110. This allows a user to insert the implant 100 into a body of a patient using a cannula having an inner diameter approximately equal to the maximum outer diameter of the bone screw 110. Said another way, the deformable portion 172 of the retention member 170 does not extend radially beyond the outer boundary of the bone screw 110 when the retention member 170 is in the first configuration. Similarly stated, when the retention member 170 is in the first configuration, an outer surface 171 of the retention member 170 is substantially aligned with an outer surface of the bone screw 110. Said another way, when the retention member 170 is in the first configuration, the outer surface 171 of the retention member 170 and the outer surface of the bone screw 110 form a substantially continuous surface. When viewed in a two-dimensional cross-section, the outer surface 171 of the retention member 170 is substantially parallel to the outer surface of the bone screw 110. Moreover, the outer surface 171 of the retention member 170 is substantially parallel to (or curved about) the longitudinal axis A_{L}.

The retention member 170 can be moved from the first configuration to the second configuration, for example, by applying mechanical forces to the retention member 170 via an insertion tool (not shown). The insertion tool can be a medical device similar to those disclosed in U.S. patent application Ser. No. 12/112,658, entitled “Apparatus and Methods for Inserting Facet Screws,” filed Apr. 30, 2008, which is incorporated herein by reference in its entirety.

The retention member 170 is moved between the first configuration (FIG. 1) and the second configuration (FIG. 2) by deforming the deformable portion 172 of the retention member 170. When the retention member 170 is in the second configuration, the retention member 170 has an outer diameter that is greater than the maximum outer diameter of the bone screw 110. Said another way, when the retention member 170 is in the second configuration, the deformable portion 172 of the retention member 170 extends radially beyond the outer boundary of the bone screw 110. Similarly stated, when the retention member 170 is in the second configuration (FIG. 2), the outer surface 171 of the retention member 170 is no longer aligned with the outer surface of the bone screw. When viewed in a two-dimensional cross-section, a portion of the outer surface 171 of the retention member 170 is substantially normal to and/or non-parallel to the outer surface of the bone screw 110 and/or the longitudinal axis A_{L}.

In use, the implant 100 is inserted into a body of a patient when the retention member 170 is in the first configuration. An insertion device, such as those described in further detail herein can be used to aid the insertion. The implant 100 can be attached to the insertion device such that longitudinal movement of the implant relative to the insertion device is limited.

When the retention member 170 is in the first configuration, the implant 100 can be inserted into a body of a patient percutaneously through a cannula. As stated above, when the retention member 170 is in the first configuration, the retention member 170 has an outer diameter that is approximately equal to the maximum outer diameter of the bone screw 110. This arrangement minimizes the inner diameter of the cannula needed to insert the implant 100 within the body of the patient. In some embodiments, for example, such a cannula has a size of between 3 mm and 25 mm. In other embodiments, the size of the cannula is approximately 5 mm. Minimizing the inner diameter of the cannula results in a smaller skin incision, less tissue distraction, can reduce patient recovery time, and minimizes the likelihood of the cannula interfering with other portions of the patient's anatomy, such as bone tissue. For example, in some embodiments, the implant can be inserted into a body of a patient through a skin incision having a length of between 3 mm and 25 mm. In other embodiments, for example, the skin incision has a length of approximately 5 mm.

In some embodiments, a guide wire and/or stylet is used to position the distal end of the cannula adjacent to bone tissue (not shown in FIGS. 1 and 2) where the implant 100 is to be inserted such that when the implant 100 is inserted through the cannula, the threaded portion 122 of the distal end portion 120 of the bone screw 110 is disposed adjacent the bone tissue where the implant 100 is to be inserted. For example, in some embodiments, the threaded portion 122 is positioned adjacent a facet joint of a spinal column.

The threaded portion 122 of the distal end portion 120 of the bone screw 110 can then be advanced into a bone tissue of a patient. In some embodiments, for example, the bone screw 110 can be inserted across a facet joint of a spinal column to fuse and/or limit the motion of the facet joint. In other embodiments, the bone screw can be used as a pedicle screw. In some an embodiment, rotating the bone screw 110 in a first direction will cause the bone screw 110 to define a passageway into the bone tissue. In this manner, the bone screw 110 is inserted into the bone tissue.

The bone screw 110 of the implant 100 is placed within the bone tissue of the patient such that the retention member 170 is not disposed within the bone tissue. In some embodiments, the entire bone screw 110 of the implant 100 is substantially disposed within the bone tissue such that a distal edge of the retention member 170 is substantially aligned with an outer surface of the bone tissue. In other embodiments, the proximal end portion of the bone screw 110 is not entirely disposed within the bone tissue. For example, in some procedures, only the distal end portion of the bone screw 110 can be disposed within the bone tissue.

After the bone screw 110 is disposed within the bone tissue, the deformable portion 172 of the retention member 170 can be moved from its first configuration to its second configuration. In some embodiments, the retention member 170 can be moved without moving the bone screw 110 with respect to the bone tissue. Similarly stated, in some embodiments, the retention member 170 can be moved from its first configuration to its second configuration is independent of advancing the bone screw 110 into the bone tissue. In some embodiments, the insertion tool can be used to both maintain the position of the bone screw 110 with respect to the bone tissue and deform the deformable portion 172 of the retention member 170, moving it to its second configuration.

When the retention member 170 is moved from its first configuration to its second configuration the deformable portion 172 of the retention member 170 is deformed such that the deformable portion 172 of the retention member 170 abuts a portion of the bone tissue. Thus, when in the second configuration, the deformable portion 172 contacts an area of the bone tissue surrounding the entry site of the bone screw 110 thereby distributing the compression load applied by the bone screw 110 and/or the retention member 170 over the outer surface of the bone tissue. For example, when the bone screw 110 is inserted across a facet joint such that the facet joint is fixated, the retention member 170 spreads the force exerted by the facet joint on the bone screw 110 over an extended surface area such that the joint is more securely maintained and the bone tissue near the retention member does not collapse and/or is not crushed. In this manner, the retention member 170 acts as a washer.

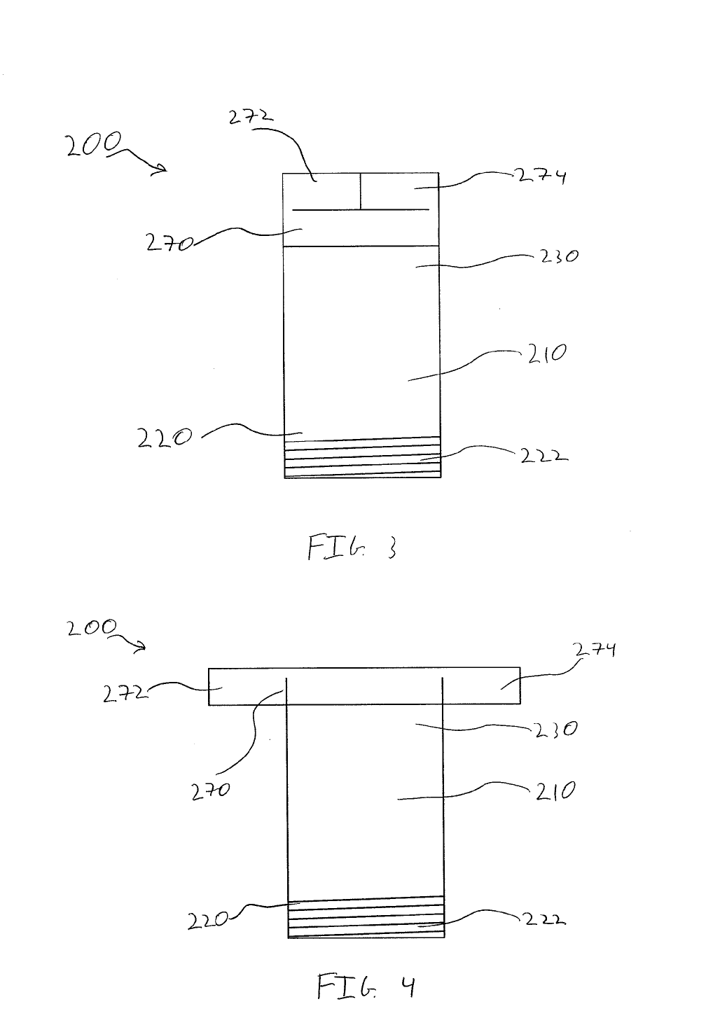

Although the retention member 170 shown in FIGS. 1 and 2 includes a single deformable portion 172, in other embodiments an implant can include a retention member with multiple deformable portions. For example, FIGS. 3 and 4 show an implant 200 including a retention member 270 having a first deformable portion 272 and a second deformable portion 274. Similar to the deformable portion 172 of the retention member 170, the first deformable portion 272 and the second deformable portion 274 of the retention member 270 are configured to plastically deform when moved from a first configuration (FIG. 3) to a second configuration (FIG. 4).

The implant 200 also includes a bone screw 210 having a proximal end portion 230 and a distal end portion 220. The proximal end portion 230 of the bone screw 210 is coupled to the retention member 270 such that movement of the retention member 270 with respect to the bone screw 210 is limited. For example, in some embodiments, the retention member 270 can be fixedly coupled to the bone screw 210. The distal end portion 220 of the bone screw 210 has a threaded portion 222. The distal end portion 222 is configured to be inserted into a bone of a patient (not shown in FIGS. 3 and 4). When the bone screw 210 is rotated, the threaded portion 222 can be advanced into the bone of the patient.

When the retention member 270 is in the first configuration (FIG. 3), the retention member 270 has an outer diameter that is approximately equal to the maximum outer diameter of the bone screw 210. This allows a user to insert the medical implant 200 into a body of a patient using a cannula having an inner diameter approximately equal to the outer diameter of the bone screw 210. Said another way, the deformable portions 272, 274 of the retention member 270 do not extend radially beyond the outer boundary the bone screw 210.

The retention member 270 is moved from the first configuration (FIG. 3) to the second configuration (FIG. 4) by deforming the deformable portions 272, 274 of the retention member 270. When the retention member 270 is in the second configuration, the retention member 270 has an outer diameter that is greater than the maximum outer diameter of the bone screw 210. Said another way, when the retention member 270 is in the second configuration, the deformable portions 272, 274 extend radially beyond the outer boundary of the bone screw 210.

In use, the implant 200 can be inserted into a bone tissue of a patient when the retention member 270 is in the first configuration. The method for inserting the implant 200 is similar to the method for inserting the implant 100, described above. After the bone screw 210 is disposed within the bone tissue, the retention member 270 can be moved from the first configuration to the second configuration by deforming the deformable portions 272, 274 of the retention member 270. In some embodiments, an insertion tool can be used to deform the deformable portions 272, 274 of the retention member 270 and/or to maintain the position of the bone screw 210 with respect to the bone tissue while the deformable portions 272, 274 of the retention member 270 are deformed.

The deformable portions 272, 274 of the retention member 270 can be deformed such that the deformable portions 272, 274 of the retention member 270 abut a surface of the bone tissue. In some embodiments, when the deformable portions 272, 274 are deformed, a portion of the deformable portions 272, 274 can exert a compression force on the surface of the bone tissue. Thus, when in the second configuration, the deformable portions 272, 274 of the retention member 270 distribute and/or apply the compression force over the outer surface of the bone tissue. In this manner, when in the second configuration, the retention member 270 helps maintain the facet joint in the fixated and/or compressed position. Similarly stated, when in the second configuration, the retention member 270 limits movement of the bone tissue relative to the bone screw 210. Moreover, by distributing the load over the area of the bone tissue, the structural integrity of the bone tissue at the interface between the implant 200 and the bone tissue is enhanced. Similarly stated, by distributing the load over the area of the bone tissue, the bone tissue near the retention member 270 is resistant to collapsing and/or is being crushed by the compression force. Additionally, having a first deformable portion 272 and a second deformable portion 274 increases the surface area of the retention member 270 that contacts the bone tissue, further spreading the force exerted by the facet joint on the bone screw 210.

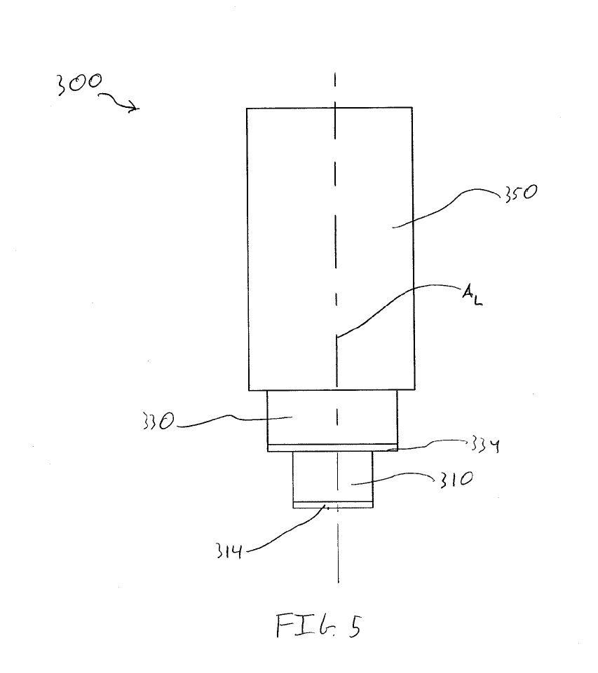

FIG. 5 is a schematic illustration of a tool 300 used to deliver a medical implant into a body of a patient, according to an embodiment. The tool 300 can be used, for example, to insert the implants 100, 200, as described above. The tool 300 includes a first shaft 310, a second shaft 330, and an actuator 350. The first shaft 310 is disposed within a lumen (not shown) defined by the second shaft 330 and is configured to rotate about a longitudinal axis A_{L} with respect to the second shaft 330 and the actuator 350.

The first shaft 310 includes an engagement portion 314 configured to engage a first engagement portion of an implant (not shown in FIG. 5). The engagement portion 314 of the first shaft 310 is configured to prevent the implant from moving with respect to the tool 300 along the longitudinal axis A_{L}. Additionally, the first engagement portion of the implant engages the engagement portion 314 of the first shaft 310 of the tool 300 such that the implant is releasably coupled to the tool 300.

The engagement portion 314 of the first shaft 310 can include any mechanism configured to releasably couple the implant to the tool 300. In some embodiments, for example, the engagement portion 314 of the first shaft 310 can include a threaded connector configured to mate with a threaded connector of the first engagement portion of the implant. In such an embodiment, when the first shaft 310 is rotated with respect to the implant about the longitudinal axis A_{L} in a first direction (e.g., clockwise), the threaded connector of the engagement portion 314 of the first shaft 310 can engage the threaded connector of the implant and couple the implant to the tool 300. Said another way, the threaded connector of the engagement portion 314 of the tool 300 can screw into the threaded connector of the first engagement portion of the implant. When the first shaft 310 is rotated with respect to the implant about the longitudinal axis A_{L} in a second direction, opposite the first direction (e.g., counter-clockwise), the threaded connector of the engagement portion 314 of the first shaft 310 is configured to release the threaded connector of the first engagement portion of the implant. In this manner, the implant can be uncoupled from the tool 300.

In other embodiments, a snap-fit connector can be used. In such an embodiment, the engagement portion of the first shaft includes one or more protrusions and a first engagement portion of an implant includes one or more detents configured to releasably receive the protrusions. In other embodiments, the engagement portion of the first shaft can include the one or more detents and the first engagement portion of the implant can include the one or more protrusions. In yet other embodiments, the implant can be releasably coupled to the tool using a clip, a magnet, and/or the like.

The second shaft 330 is disposed within a lumen (not shown) defined by the actuator 350 and is configured to rotate about the longitudinal axis A_{L} with respect to the actuator 350. In some embodiments, the second shaft 330 is also configured to rotate with respect to the first shaft 310. The second shaft 330 includes an engagement portion 334 configured to engage a second engagement portion of an implant. The engagement portion 334 of the second shaft 330 is configured to engage the second engagement portion of the implant such that rotation of the second shaft 330 of the tool 300 causes a portion of the implant to similarly rotate.

The engagement portion 334 of the second shaft 330 can include any mechanism configured to engage and rotate the second engagement portion of the implant. In some embodiments, for example, the engagement portion 334 of the second shaft 330 can include one or more protrusions configured to engage one or more notches and/or detents defined by the second engagement portion of the implant. In such an embodiment, as the protrusions of the engagement portion 334 of the second shaft 330 rotate, they contact the second engagement portion of the implant and similarly rotate a portion of the implant. In other embodiments, the engagement portion of the second shaft can include the one or more notches and/or detents and the first engagement portion of the implant can include the one or more protrusions. In yet other embodiments, the engagement portion of the second shaft of the tool can include a clip, a snap-fit connector, and/or the like to rotatably engage a portion of the implant.

The actuator 350 of the tool 300 surrounds at least a portion of the first shaft 310 of the tool 300 and at least a portion of the second shaft 330 of the tool 300. The actuator 350 is configured to move with respect to the first shaft 310 and the second shaft 330 along the longitudinal axis A_{L}. The actuator 350 is substantially rigid such that it can deform a portion of a retention member of an implant when the actuator 350 contacts the retention member of the implant.

In use, the implant is coupled to the tool 300 by attaching the engagement portion 314 of the first shaft 310 of the tool 300 to the first engagement portion of the implant. This prevents movement of the implant with respect to the tool 300 along the longitudinal axis A_{L}. The engagement portion 334 of the second shaft 330 of the tool 300 can then be positioned such that it engages the second engagement portion of the implant.

The implant is inserted into a body of a patient such that a distal portion of the implant is disposed adjacent to the bone tissue of the patient. The second shaft 330 of the tool 300 is then rotated about the longitudinal axis A_{L} in a first direction (e.g., clockwise). This causes a bone screw portion of the implant to be advanced into the bone tissue. After the implant is placed such that at least a portion of the bone screw portion of the implant is disposed within the bone tissue, the actuator 350 can be moved along the longitudinal axis A_{L} in a distal direction while the position of the bone screw portion of the implant within the bone tissue is maintained by maintaining the position of the first shaft 310 of the tool 300. The movement of the actuator 350 relative to the implant causes a distal end portion of the actuator 350 to contact a proximal end portion of the retention member of the implant. The force of the movement of the actuator 350 in the distal direction causes a deformable portion of the retention member of the implant to deform.

In some embodiments, when the actuator 350 is moved to exert a force on the deformable portion of the retention member, the user can exert a substantially equal and opposite force on the bone screw portion of the implant by via the first shaft 310 of the tool 300 (e.g., by pulling proximally on the first shaft 310 of the tool 300 while the first shaft is coupled to the implant). This causes a substantially zero net force to be exerted on the distal end portion of the bone screw when the deformable portion of the retention member is deformed. Similarly stated, the bone screw portion of the implant does not move with respect to the bone tissue when the actuator 350 exerts a force on the retention member. In this manner, the force applied by the actuator 350 to the implant is not transferred to the portion of the bone screw that is disposed within the bone tissue. In this manner, the structural integrity of the bone tissue within which the bone screw is disposed can be maintained. For example, in some embodiments, this arrangement can prevent a threaded opening within the bone tissue from being stripped when the actuator 350 applies a compression force. Thus, the bone screw portion of the implant does not move with respect to the bone tissue and the bone tissue is not crushed.

After the deformable portion is deformed such that it contacts the bone tissue surrounding the insertion site of the implant, the implant can be released from the tool 300 and the tool 300 can be removed from the body of the patient. The implant is released from the tool 300 by releasing the engagement portion 314 of the first shaft 310 of the tool 300 and the engagement portion 334 of the second shaft 330 of the tool 300 from the first engagement portion of the implant and the second engagement portion of the implant, respectively. Using a single tool such as tool 300, an implant can be inserted into a body of a patient without repeatedly inserting and removing a set of tools from the body of the patient. This minimizes the trauma and damage that can occur to the body tissue of the patient.

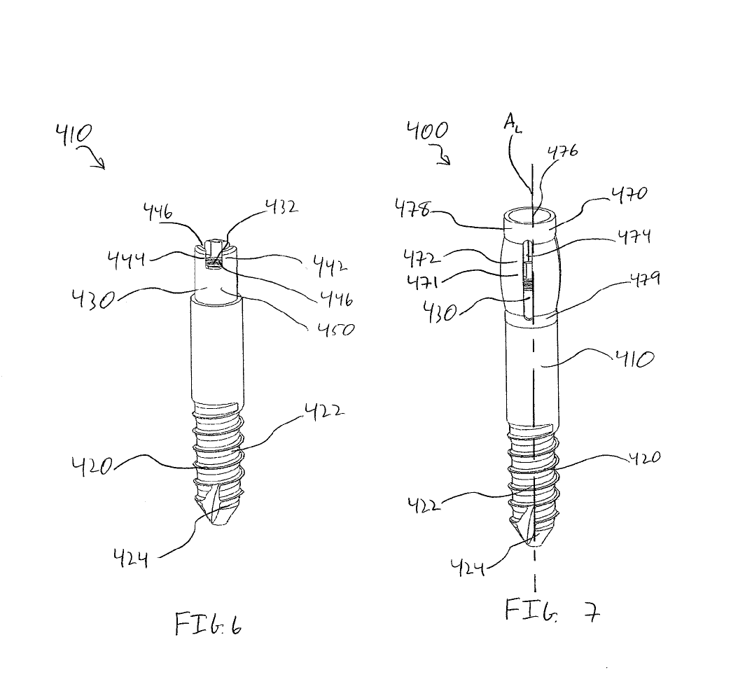

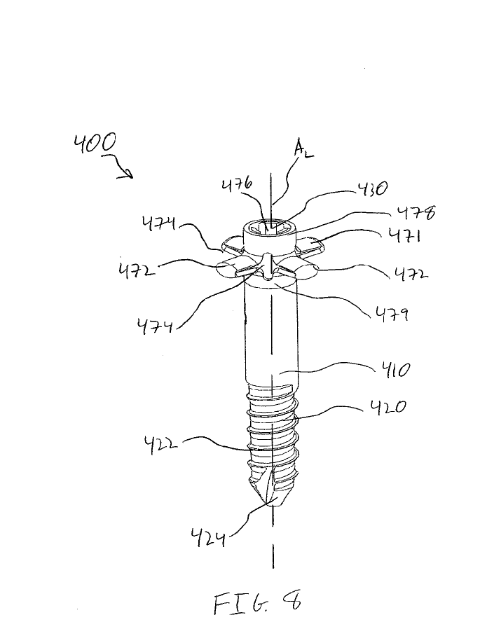

FIGS. 7-8 show an implant 400 having a retention member 470 in a first configuration and a second configuration respectively, according to an embodiment. The implant 400 includes a bone screw 410 and a retention member 470. FIG. 6 shows the bone screw 410 of the implant 400.

The bone screw 410 of the implant 400 includes a distal end portion 420 and a proximal end portion 430. The distal end portion 420 of the bone screw 410 includes a threaded portion 422 and a self-tapping portion 424. The distal end portion 420 of the bone screw 410 is configured to be inserted into a bone tissue of a patient. The self-tapping portion 424 of the distal end portion 420 of the bone screw 410 allows the bone screw 410 to be inserted into a bone tissue without forming a hole in the bone tissue prior to insertion. Said another way, the self-tapping portion 424 of the distal end portion 420 of the bone screw 410 produces a hole in the bone tissue for the bone screw 410 to enter the bone tissue. As the bone screw 410 is rotated, the threaded portion 422 advances the bone screw 410 into the bone tissue of the patient, as further described herein. In other embodiments, the bone screw is not self-tapping and a hole or passageway is defined in the bone tissue prior to placing the bone screw within the bone tissue.

Although the bone screw 410 is shown in FIGS. 6-8 as having a threaded portion 422 having a cross sectional size (e.g., an outer diameter) that is substantially equal to a cross-sectional size (e.g., an outer diameter) of other portions of the bone screw 410 (e.g., the shank), in other embodiments, the threaded portion 422 can have a cross sectional size that is greater than the cross-sectional size of other portions of the implant. In such an embodiment, for example, a non-threaded shank of the bone screw can fit easily within the hole formed by the threaded portion.

The proximal end portion 430 of the bone screw 410 includes a first engagement portion 432, a second engagement portion 442, and a recessed portion 450. The recessed portion 450 is configured to be received within a lumen 476 defined by the retention member 470 (see e.g., FIG. 7). In this manner, the retention member 470 can be coupled to the proximal end portion 430 of the bone screw 410 without increasing the outer diameter of the implant 400. The retention member can be coupled to the proximal end portion of the bone screw by any suitable means such as, for example, a clip, an adhesive, a snap-fit connection, a weld joint and/or the like. In still other embodiments, the retention member can be monolithically formed with the bone screw. In some embodiments, rotational movement of the retention member 470 relative to the bone screw 410 is prevented.

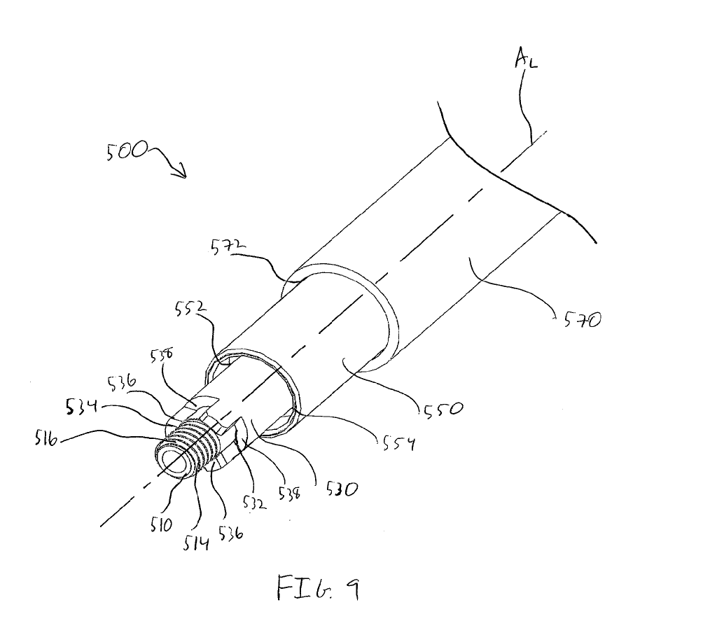

The first engagement portion 432 of the proximal end portion 430 of the bone screw 410 is configured to releasably couple the implant 400 to an insertion device. For example, the first engagement portion 432 of the proximal end portion 430 of the bone screw 410 is configured to receive an engagement portion 514 of a first shaft 510 of an insertion tool 500, as described below with reference to FIG. 9. As shown in FIG. 6, the first engagement portion 432 includes a plurality of threads. The plurality of threads of the first engagement portion 432 are configured to receive a male threaded portion 516 of the engagement portion 514 of the first shaft 510 of an insertion tool 500, as described herein. In other embodiments, any mechanism configured to releasably couple the implant to the tool can be used, such as, for example, a snap-fit connector, a clip, a magnet, and/or the like.

The second engagement portion 442 of the proximal end portion 430 of the bone screw 410 includes a plurality of protrusions 444 that define a plurality of notches 446. Although FIG. 6 shows the second engagement portion 442 of the proximal end portion 430 of the bone screw 410 as having three protrusion 444 defining three notches 446, in other embodiments, the second engagement portion of the proximal end portion can have any number of notches and/or protrusions.

The second engagement portion 442 of the proximal end portion 430 of the bone screw 410 is configured to engage an engagement portion 534 of a second shaft 530 of the insertion tool 500, as described herein with reference to FIG. 9. The second engagement portion 442 is configured to engage the insertion tool 500 such that rotation of the bone screw 410 relative to the insertion tool 500 is prevented. Thus, when the second engagement portion 442 is engaged with the engagement portion 534 of the second shaft 530, rotation of the second shaft 530 of the insertion tool causes the bone screw 410 (including the threaded portion 422 of the distal end portion 420) to similarly rotate.

FIG. 7 shows the implant 400 with the retention member 470 coupled to the bone screw 410 and in the first configuration. The retention member 470 includes a proximal end portion 478, a distal end portion 479, and a deformable portion 472. The retention member 470 also defines a plurality of elongated openings 474 and a lumen 476 configured to receive the recessed portion 450 of the bone screw 410. The recessed portion 450 of the bone screw 410 allows the distal end portion 479 of the retention member 470 to be disposed about the proximal end portion 430 of the bone screw 410 without increasing the outer diameter of the implant 400 in the first configuration, as described in detail herein.

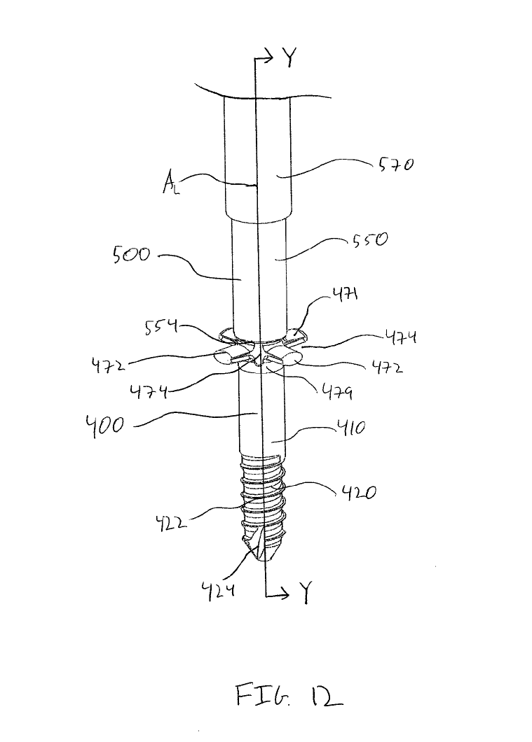

When the retention member 470 is in the first configuration (FIG. 7), the retention member 470 has an outer diameter that is approximately equal to the outer diameter of the bone screw 410. This allows a user to insert the medical implant 400 into a body of a patient using a cannula having an inner diameter approximately equal to the outer diameter of the bone screw 410. Said another way, when the retention member 470 is in the first configuration the deformable portion 472 of the retention member 470 does not extend radially beyond the outer boundary of the bone screw 410 (FIG. 7). Similarly stated, when the retention member 470 is in the first configuration, an outer surface 471 of the retention member 470 is substantially aligned with an outer surface of the bone screw 410. Said another way, when the retention member 470 is in the first configuration, the outer surface 471 of the retention member 470 and the outer surface of the bone screw 410 form a substantially continuous surface. As shown in FIG. 13, when viewed in a two-dimensional cross-section, at least a portion of the outer surface 471 of the retention member 470 is substantially parallel to the outer surface of the bone screw 410.

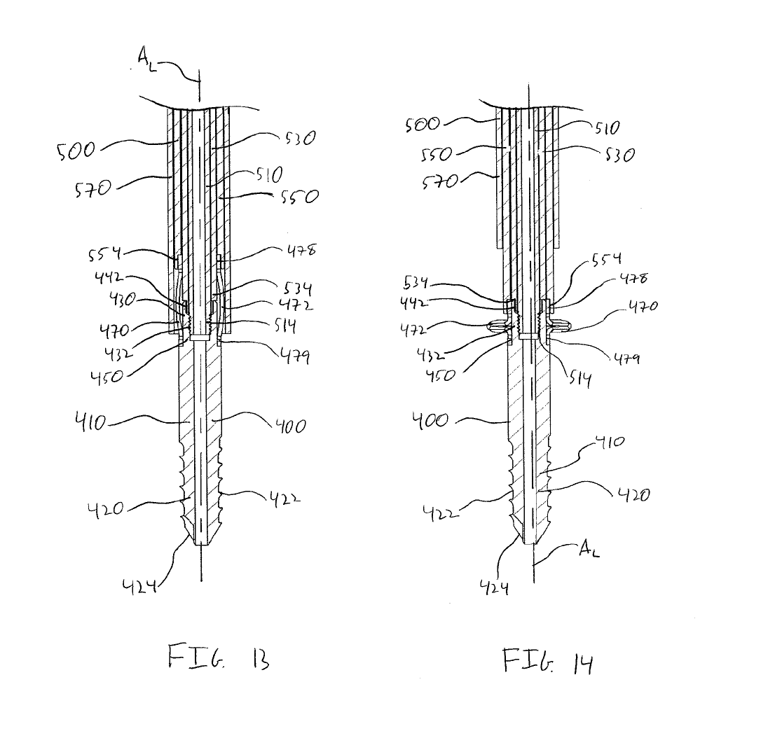

The deformable portion 472 of the retention member 470 is configured to plastically deform when moved from the first configuration (FIGS. 7 and 13) to the second configuration (FIGS. 8 and 14). In this manner, the deformable portion 472 of the retention member 470 is deformed such that the implant 400 remains in the second configuration. The elongated openings 474 and the shape of the deformable portion 472 of the retention member 470 collectively allow the retention member 470 to deform in a predetermined manner when a compression force is applied. More particularly, the deformable portion 472 is slightly curved outwardly when in the first configuration, thus causing the deformable portion 472 to expand outwardly when deformed. Although the retention member 470 is shown in FIGS. 7 and 8 as defining four elongated openings 474, the retention member can have any number of elongated openings. The number of elongated openings corresponds to the number of portions (or “legs”) of the deformable portion. For example, because the implant 400 includes four elongated openings 474, the deformable portion 472 is deformed into four separate portions when moved from the first configuration to the second configuration (see e.g., FIG. 8). The elongate openings 474 of the retention member 470 are substantially parallel to the longitudinal axis A_{L} when the retention member 470 is in the first configuration.

To move the retention member 470 from the first configuration to the second configuration, a compressive force is applied to the proximal end portion 478 of the retention member 470 while the location of the distal end portion 479 of the retention member 470 and/or the location of the bone screw 410 with respect to the bone tissue is maintained. Similarly stated, to move the retention member 470 from the first configuration to the second configuration, a first compressive force is applied to the proximal end portion 478 of the retention member 470 and a second compressive force, substantially equal and opposite to the first compressive force is applied to the distal end portion 479 of the retention member 470. The compressive force causes the proximal end portion 478 of the retention member 470 to move in a distal direction along the longitudinal axis A_{L} with respect to the bone screw 410. The movement of the proximal end portion 478 relative to the distal end portion 479 and/or the bone screw 410 causes the deformable portion 472 of the retention member 470 to deform such that a portion of the outer surface 471 of each separate portion of the deformable portion 472 extends at an angle substantially normal to the longitudinal axis A_{L} as shown in FIG. 8. Said another way, the deformable portion 472 of the retention member 470 is substantially non-linear and substantially non-parallel to the longitudinal axis A_{L} when the retention member 470 is in the second configuration.

When the retention member 470 is in the second configuration, the retention member 470 has an outer diameter that is greater than the outer diameter of the bone screw 410. Said another way, when the retention member 470 is in the second configuration, the deformable portion 472 of the retention member 470 extends radially beyond the outer boundary of the bone screw 410 (see e.g., FIG. 8). Similarly stated, when the retention member 470 is in the second configuration, the outer surface 471 of the retention member 470 is not longer aligned with the outer surface of the bone screw 410.

The proximal end portion 478 of the retention member 470 is configured to receive a tool configured to aid in the insertion of the implant 400 into the bone tissue of a patient. For example, FIG. 9 shows a portion of a tool 500 used to deliver, insert and/or actuate the implant 400, within a body of a patient, according to an embodiment. The tool 500 can be inserted through the lumen 476 defined by the retention member 470 such that the tool 500 engages the first engagement portion 432 and the second engagement portion 442 of the proximal end portion 430 of the bone screw 410, as described below.

The tool 500 includes a first shaft 510, a second shaft 530, an actuator 550, and a sheath 570. The first shaft 510 is disposed within a lumen 532 defined by the second shaft 530 and is configured to rotate about a longitudinal axis A_{L} with respect to the second shaft 530 and the actuator 550.

The first shaft 510 includes an engagement portion 514 configured to engage the first engagement portion 432 of the proximal end portion 430 of the implant 400. The engagement portion 514 of the first shaft 510 is configured to prevent the implant 400 from moving with respect to the tool 500 in a direction parallel to the longitudinal axis A_{L}. Additionally, the first engagement portion 432 of the implant 400 engages the engagement portion 514 of the first shaft 510 of the tool 500 such that the implant is releasably coupled to the tool 500.

The engagement portion 514 of the first shaft 510 includes a threaded portion 516 configured to mate with the threaded portion of the first engagement portion 432 of the implant 400. Thus, when the first shaft 510 is rotated with respect to the second shaft 530 about the longitudinal axis A_{L} in a first direction (e.g., clockwise), the threaded portion 516 of the engagement portion 514 of the first shaft 510 can engage the threaded portion of the first engagement portion 432 of the implant 400 and couple the implant 400 to the tool 500. Said another way, the threaded portion 516 of the engagement portion 514 of the tool 500 can screw into the threaded portion of the first engagement portion 432 of the implant 400. When the first shaft 510 is rotated with respect to the second shaft 530 about the longitudinal axis A_{L} in a second direction, opposite the first direction (e.g., counter-clockwise), the threaded portion 516 of the engagement portion 514 of the first shaft 510 is configured to release the threaded portion of the first engagement portion 432 of the implant 400. In this manner, the implant 400 can be uncoupled from the tool 500.

The second shaft 530 of the tool 500 is disposed within a lumen 552 defined by the actuator 550 and is configured to rotate about the longitudinal axis A_{L} with respect to the actuator 550. In some embodiments, the second shaft 530 is also configured to rotate with respect to the first shaft 510. The second shaft 530 includes an engagement portion 534 configured to engage the second engagement portion 442 of the implant 400 such that rotation of the second shaft 530 of the tool 500 causes a portion of the implant 400 to similarly rotate.

The engagement portion 534 of the second shaft 530 includes one or more protrusions 536, which define one or more notches 538. The protrusions 536 of the engagement portion 534 of the second shaft 530 are configured to engage the notches 446 defined by the protrusions 444 of the second engagement portion 442 of the implant 400. Similarly, the protrusions 444 of the second engagement portion 442 of the implant 400 are configured to fit within the notches 538 defined by the protrusions 536 of the engagement portion 534 of the second shaft 530. As the protrusions 536 of the engagement portion 534 of the second shaft 530 rotate, they contact the protrusions 444 of the second engagement portion 442 of the implant 400 and rotate a portion of the implant 400.

In other embodiments, the engagement portion of the second shaft of the insertion tool can engage the second engagement portion of the proximal end portion of the bone screw in any manner that causes the bone screw to rotate when the second shaft of the insertion tool rotates. For example, a clip, a snap-fit connector, and/or the like can be used.

The actuator 550 of the tool 500 surrounds at least a portion of the first shaft 510 and the second shaft 530, and is disposed within a lumen 572 defined by the sheath 570. The actuator 550 is configured to move with respect to the first shaft 510 and the second shaft 530 in a direction parallel to the longitudinal axis A_{L}. The actuator 550 is substantially rigid such that it can move the retention member 470 of the implant 400 from its first configuration to its second configuration when the actuator 550 contacts the retention member 470 of the implant. Similarly stated the actuator 550 is configured to exert a compressive force on the retention member 470 of sufficient magnitude to cause the deformable portion 472 of the retention member 470 to deform.

The sheath 570 of the tool 500 is disposed about the actuator 550 of the tool 500. The sheath 570 of the tool 500 is configured to move with respect to the actuator 550, the first shaft 510, and the second shaft 530 in a direction substantially parallel to the longitudinal axis A_{L}. In a first position, the sheath 570 covers at least a portion of the retention member 470 of the implant 400. In this manner, the sheath 570 can prevent the deformable portion 472 of the retention member 470 from moving from its first configuration to its second configuration. In a second position, the sheath 570 is not disposed about the retention member 470 of the implant 400. The sheath 570 of the tool 500 can also be used as a depth indicator to indicate to the user when the implant 400 is sufficiently disposed within the bone tissue of a patient, as further described herein.

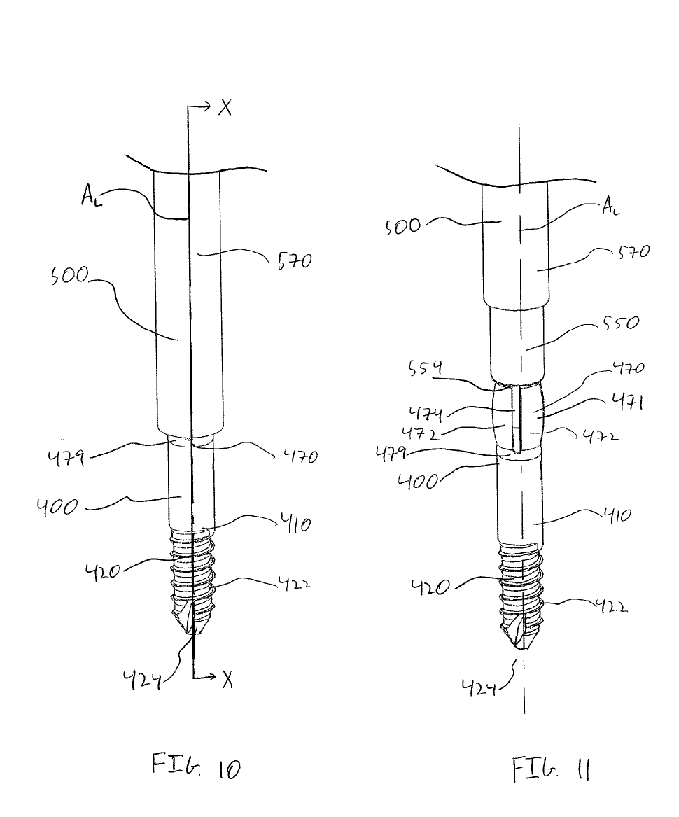

FIGS. 10, 11, and 12 show perspective views of the implant 400 in various configurations connected to the tool 500 in various positions. The implant 400 is coupled to the tool 500 by attaching the engagement portion 514 of the first shaft 510 of the tool 500 to the first engagement portion 432 of the implant 400 (see also FIGS. 6 and 9). This prevents movement of the implant 400 with respect to the tool 500 along the longitudinal axis A_{L}. The engagement portion 534 of the second shaft 530 of the tool 500 can then be positioned such that it engages the second engagement portion 442 of the implant 400 (see also FIGS. 6 and 9). Once the engagement portion 534 of the second shaft 530 of the tool 500 engages the second engagement portion 442 of the implant 400, the sheath 570 of the tool 500 can be positioned such that it extends over at least a portion of the retention member 470 of the implant 400 (see FIG. 10). A cross-sectional view of the sheath 570 disposed over the retention member 470 can be seen in FIG. 13.

The implant 400 is then inserted into a body of a patient such that the distal end portion 420 of the bone screw 410 of the implant 400 is disposed adjacent the bone tissue of the patient. For example, the bone screw 410 can be disposed adjacent a facet joint. The second shaft 530 of the tool 500 is then rotated about the longitudinal axis A_{L} in a first direction (e.g., clockwise). This causes the threaded portion 422 of the distal end portion 420 of the bone screw 410 to similarly rotate and advance into the bone tissue. In some embodiments, for example, a portion of the bone screw 410 can be inserted within a passageway defined by an inferior vertebra and a superior vertebra such that a central portion of the bone screw 410 crosses a facet joint between the inferior vertebra and the superior vertebra. For example, when the bone screw 410 is inserted across a facet joint the facet joint is fixated, brought together and/or compressed by the bone screw 410.

The self-tapping portion 424 enables the bone screw 410 to enter the bone tissue without first drilling a hole in the bone tissue. In this manner, the bone screw 410 can be inserted into the bone tissue of the patient until a distal end of the sheath 570 contacts the bone tissue. When the distal end of the sheath 570 contacts the bone tissue, the user is alerted that the bone screw 410 is sufficiently disposed within the bone tissue such that when the retention member 470 is deformed, a portion of the retention member 470 will contact the surface of the bone tissue.

The sheath 570 of the tool 500 can be moved with respect to the implant 400 in a proximal direction such that the sheath 570 no longer extends over the retention member 470 of the implant 400 (FIG. 11). This exposes the distal end portion 479 of the retention member 470 to the edge of the bone tissue. After the sheath 570 of the tool 500 is moved such that it does not extend over the retention member 470, the actuator 550 of the tool 500 can be used to deform the deformable portion 472 of the retention member 470 of the implant 400. In this manner, the deformable portion 472 of the retention member 470 can be deformed using the actuator independent of advancing the bone screw 410 into the bone tissue, described above.

To deform the deformable portion 472 of the retention member 470, a user maintains the position of the first shaft 510 of the tool 500 while moving the actuator 550 with respect to the implant 400 along the longitudinal axis A_{L} in a distal direction. In some embodiments, when the actuator 550 is moved to exert a force on the deformable portion 472 of the retention member 470, the user can exert a substantially equal and opposite force on the bone screw 410 of the implant 400 by via the first shaft 510 of the tool 500 (e.g., by pulling proximally on the first shaft 510 of the tool 500 while the first shaft is coupled to the implant). This causes a distal end portion of the actuator 550 to contact the proximal end portion 478 of the retention member 470 of the implant 400. The actuator 550 exerts a compressive force on the proximal end portion 478 of the retention member 470 which causes the retention member 470 to move from its first configuration to its second configuration (FIG. 12).

Moreover, moving the actuator 550 while maintaining the position of the first shaft 510 causes a substantially zero net force to be exerted on the distal end portion of the bone screw when the deformable portion of the retention member is deformed. Similarly stated, the bone screw 410 of the implant 400 does not move with respect to the bone tissue when the actuator 550 exerts a force on the retention member 470. In this manner, the force applied by the actuator 550 to the implant is not transferred to the distal end portion 420 of the bone screw 410. In this manner, the structural integrity of the bone tissue within which the bone screw 410 is disposed can be maintained. For example, in some embodiments, this arrangement can prevent a threaded opening within the bone tissue from being stripped when the actuator 550 applies a compression force to the retention member 450.

After the retention member 470 is in its second configuration, the deformable portion 472 of the retention member 470 contacts and/or exerts a force on the bone tissue surrounding the insertion site of the implant 400. When the deformable portion 472 of the retention member 470 contacts the bone tissue surrounding the insertion site of the bone screw 410, the compression load applied by the bone screw 410 is distributed over a larger area than would be the case without the retention member 470. For example, when the bone screw 410 is inserted across a facet joint such that the facet joint is fixated, brought together and/or compressed by the bone screw 410, the retention member 470 helps maintain the facet joint in the fixated and/or compressed position. The retention member 470 spreads the force exerted by the facet joint on the bone screw 410 over an extended area such that the joint is more securely maintained in the fixated, fused and/or compressed position and the bone near the retention member 470 does not collapse and/or is not crushed. In this manner, the retention member 470 acts as a washer.

The implant 400 can then be released from the tool 500 and the tool 500 can be removed from the body of the patient. The implant 400 is released from the tool 500 by releasing the engagement portion 514 of the first shaft 510 of the tool 500 from the first engagement portion 432 of the implant 400 by unscrewing the first shaft 510 and releasing the engagement portion 534 of the second shaft 530 of the tool 500 from the second engagement portion 442 of the implant 400 by proximal movement of the second shaft 530.

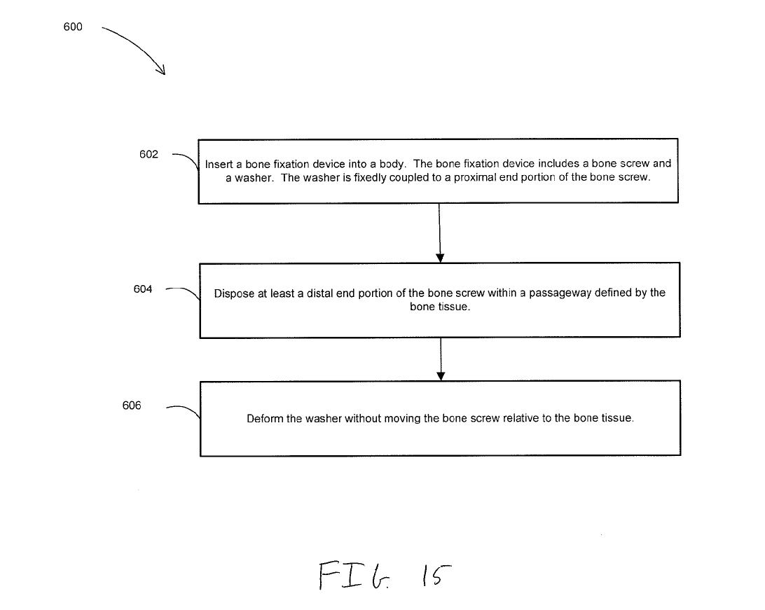

FIG. 15 is a flow chart of a method 600 of implanting an implant within a body of a patient, according to an embodiment. The method 600 includes inserting a bone fixation device into a body, at 602. The bone fixation device includes a bone screw and a retention member. The retention member is fixedly coupled to a proximal end portion of the bone screw. A distal end portion of the bone screw is then disposed within a passageway defined by the bone tissue, at 604. The retention member is then deformed without moving the bone screw relative to the bone tissue, at 606. In such a manner, the implant can be inserted into a bone tissue within a body of a patient without repeatedly inserting and removing the insertion tool.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Although the bone screws are shown and described above as being substantially cylindrical (i.e., as having a circular-cross section), in other embodiments a portion of a bone screw can have a non-circular cross-sectionals shape. In some embodiments, for example, a portion of the bone screw (e.g., a proximal end portion, a shank portion, or the like) can have any shape such as, for example, a polygonal cross sectional shape, an oval cross-sectional shape, or a circular cross sectional shape. Similarly, the retention member can have any cross-sectional shape, such as, for example, a polygonal cross sectional shape, an oval cross-sectional shape, or a circular cross sectional shape.

Although the bone fixation devices are shown and described above as including a bone screw that can be threadedly advanced into a bone tissue, in other embodiments, a bone fixation device can include a pin that is devoid of threads. For example, in some embodiments, a bone fixation device can include a pin configured to be inserted into a bone tissue. The pin can be inserted by exerting a longitudinal force on a proximal end of the pin such that the pin is inserted into the bone tissue. In this manner, the pin can be advanced into the bone tissue without rotating or “screwing” the pin into the bone tissue.

Although various embodiments have been described as having particular features and/or combinations of components, other embodiments are possible having a combination of any features and/or components from any of embodiments where appropriate.

What is claimed is:

1. An apparatus, comprising:
a bone screw having a proximal end portion and a distal end portion, the distal end portion including a threaded portion; and
a retention member coupled to the proximal end portion of the bone screw such that movement of the retention member relative to the bone screw is limited, the retention member configured to deform when moved from a first configuration to a second configuration.

2. The apparatus of claim 1, wherein the bone screw defines a lumen therethrough.

3. The apparatus of claim 1, wherein the proximal end portion of the bone screw includes an engagement portion configured to engage an insertion tool such that rotation of the insertion tool results in rotation of the bone screw.

4. The apparatus of claim 1, wherein the proximal end portion of the bone screw includes an engagement portion configured to be coupled to an insertion tool such that longitudinal movement of the bone screw relative to the insertion tool in a distal direction is prevented.

5. The apparatus of claim 1, wherein the proximal end portion of the bone screw includes a first engagement portion and a second engagement portion, the first engagement portion configured to engage a first shaft of an insertion tool such that rotation of the first shaft results in rotation of the bone screw, the second engagement portion configured to be coupled to a second shaft of the insertion tool such that longitudinal movement of the bone screw relative to the second shaft in a distal direction is prevented.

6. The apparatus of claim 1, wherein the retention member is coupled to the proximal end portion of the bone screw such that rotational movement of the retention member relative to the bone screw is prevented.

7. The apparatus of claim 1, wherein a distal end portion of the retention member is fixedly coupled to the proximal end portion of the bone screw.

8. The apparatus of claim 1, wherein a surface of the retention member is disposed apart from a longitudinal axis of the bone screw by a first distance when in the first configuration, the surface of the retention member is disposed apart from the longitudinal axis of the bone screw by a second distance when in the second configuration, the second distance greater than the first distance.

9. The apparatus of claim 1, wherein a central portion of the retention member defines a plurality of elongated openings, the plurality of openings being substantially parallel to a longitudinal axis of the retention member when the retention member is in the first configuration.

10. The apparatus of claim 1, wherein a surface of the retention member is substantially parallel to an outer surface of the bone screw when the retention member is in the first configuration, the surface of the retention member is substantially normal to the outer surface of the bone screw when the retention member is in the second configuration.

11. The apparatus of claim 1, wherein the retention member is configured to be moved from the first configuration to the second configuration without movement of the bone screw relative to a distal end portion of the retention member.

12. An apparatus, comprising:
a first shaft having an engagement portion configured to engage a first engagement portion of a bone fixation device such that axial movement of the bone fixation device relative to the first shaft in a distal direction is limited;
a second shaft having an engagement portion configured to engage a second engagement portion of the bone fixation device such that rotation of the second shaft results in rotation of the bone fixation device; and
an actuator movably disposed about the second shaft, the actuator configured to deform a portion of the bone fixation device when the engagement portion of the first shaft is engaged with the first engagement portion of the bone fixation device and the engagement portion of the second shaft is engaged with the second engagement portion of the bone fixation device.

13. The apparatus of claim 12, wherein:
the engagement portion of the first shaft includes a threaded portion; and
the engagement portion of the second shaft include a plurality of protrusions configured to be received within a plurality of openings defined by the bone fixation device.

14. The apparatus of claim 12, wherein the actuator is configured to move longitudinally about the second shaft to deform the portion of the bone fixation device.

15. The apparatus of claim 12, further comprising:
an indicator movably disposed about the actuator between a first position and a second position, the indicator configured to indicate a predetermined depth of insertion of the bone fixation device when in the first position.

16. The apparatus of claim 12, wherein longitudinal movement of the bone screw relative to the first shaft in a distal direction is prevented.

17. A method, comprising:
inserting a bone fixation device into a body, the bone fixation device including a bone screw and a retention member, the retention member fixedly coupled to a proximal end portion of the bone screw;
disposing at least a distal end portion of the bone screw within a passageway defined by a bone tissue; and
deforming the retention member without moving the bone screw relative to the bone tissue, the disposing being independent from the deforming.

18. The method of claim 17, wherein the inserting includes inserting the bone fixation percutaneously via a cannula having a diameter substantially the same as a diameter of the bone screw of the bone fixation device.

19. The method of claim 17, wherein:
the inserting is performed using an insertion tool coupled to the bone fixation device; and
the disposing is performed using the insertion tool without removing the insertion tool from the body after the inserting; and
the deforming is performed using the insertion tool without removing the insertion tool from the body after the disposing.

20. The method of claim 17, wherein:
the disposing includes disposing a threaded portion of the bone screw into the bone tissue until an indicator of an insertion tool contacts an outer surface of the bone tissue.

21. The method of claim 17, wherein:
the disposing includes defining the passageway within the bone tissue using the distal end portion of the bone screw.

22. The method of claim 17, wherein:
the deforming includes moving a surface of the retention member from a first position in which the surface is substantially parallel to a longitudinal axis of the bone fixation device to a second position in which the surface is substantially normal to the longitudinal axis of the bone fixation device.

23. The method of claim 17, wherein:
the deforming includes moving a surface of the retention member from a first position in which the surface is substantially parallel to a longitudinal axis of the bone fixation device to a second position in which the surface is in contact with an outer surface of the bone tissue.

24. The method of claim 17, wherein:
the bone tissue includes an inferior vertebra and a superior vertebra; and
the disposing includes disposing the portion of the bone screw within the passageway defined by the inferior vertebra and the superior vertebra such that a central portion of the bone screw crosses a facet joint between the inferior vertebra and the superior vertebra.
